(19) United States
(12) Patent Application Publication
(10) Pub. No.: US 2024/0071740 A1
(43) Pub. Date: Feb. 29, 2024

(54) METHOD OF SCALE INHIBITOR ANALYSIS

(71) Applicant: Saudi Arabian Oil Company, Dhahran (SA)

(72) Inventors: Tao CHEN, Dhahran (SA); Qiwei WANG, Dhahran (SA); Faisal ALRASHEED, Dhahran (SA)

(21) Appl. No.: 17/822,950

(22) Filed: Aug. 29, 2022

(51) Int. Cl. H01J 49/10; C08F 220/58; C09K 8/532; G01N 33/18

(52) U.S. Cl. CPC H01J 49/105; C08F 220/585; C09K 8/532; G01N 33/18; C08F 2800/10

(57) ABSTRACT

A method of analyzing the residual of a polymeric scale inhibitor in water containing SO₄²⁻, such as produced water, is disclosed. The water also contains a polymeric scale inhibitor that includes 2-acrylamino-2-methylpropane sulfonic acid (AMPS). In the method, an amount of SO₄²⁻ in a sample of the water is measured. A water soluble salt is introduced into the sample of water, and the salt removes SO₄²⁻ from the water sample resulting in a low SO₄²⁻ water sample. The low SO₄²⁻ water sample is then analyzed, and the analysis includes: determining a sulfur level in the low SO₄²⁻ water sample, and performing a thermodynamic scale prediction to calculate the amount of SO₄²⁻ ions remaining in the low SO₄²⁻ water sample. A concentration of the polymeric scale inhibitor in the water sample is then determined based on an inductively couple plasma (ICP) analysis.

### FIELD OF THE DISCLOSURE

The present disclosure relates to methods of analyzing produced water, and in particular methods for analyzing scale inhibitors present in the produced water.

### BACKGROUND OF THE DISCLOSURE

Iron sulfide surface deposition (scaling) is a persistent issue in oil and gas wells, especially for sour gas wells. These iron sulfide scales can develop as a result of ferrous ions reacting with sour gas to form iron sulfide deposits in a downhole tubular for example, which affects well deliverability, interferes with well surveillance, and restricts well intervention.

Inhibition and prevention of iron sulfide scale formation in oil and gas wells has proven to be difficult task. Many scale inhibitor compositions (e.g., polymeric scale inhibitors) have been developed, which are used in squeeze treatments to control scale deposition in wells. Scale inhibitor squeeze treatments are one of the most common and efficient methods for preventing the formation of scales in the near wellbore, tubing and topside facilities of production wells. However, for many of the scale inhibitors, high concentrations of the scale inhibitors are needed to even provide limited inhibition performance.

Recently, improved scale inhibitors have been developed which provide improved inhibition of iron sulfide scales. But even with improvement to the effectiveness of scale inhibitors, for any scale management strategy to ultimately be successful, the scale management strategy must increase the lifetime of the well to maximize oil production and minimize the number of costly well interventions.

To achieve the desired longevity for scale squeeze treatments and prevent wells from scaling, the amount of scale inhibitor in the produced fluids of the wells must be monitored to ensure that it remains at a high enough concentration to prevent scaling. As such, accurate and precise quantitative determination of the concentration of scale inhibitor in the produced fluids is vital for preventing scale formation long term.

There are a wide variety of scale inhibitor quantitative determination techniques that exist for the detection of phosphonates, phosphate esters and polymeric scale inhibitors in the oil and gas wells. These techniques include, for example: methods based upon wet chemical analysis techniques developed by HACH® and methods using HYAMINE® 1622 (benzethonium chloride) by applying fluorescence spectroscopic techniques, inductively coupled plasma—optical emission spectrometry (ICP-OES) and inductively coupled plasma—mass spectrometry (ICP-MS), ion chromatography (IC) (e.g., for phosphonate assay and where applicable phosphorous speciation), and more complex polymer assay methods such as high pressure liquid chromatography (HPLC) and mass spectroscopy (MS).

However, for certain polymeric scale inhibitors that comprise sulfur compounds or ions, there can be interference from sulfate ions (SO₄²⁻) already present in the produced water, making it difficult to make an accurate determination of the concentration of the scale inhibitor in the produced water using an ICP analysis, for example. While other methods, such as HPLC methods, can more easily discern sulfate ions from certain small molecules polymers, these other methods have difficulty detecting larger molecule polymers (e.g., molecular weight between approximately 300 kDa and 1200 kDa) accurately to a very low concentration. Difficulty in measuring the residual concentrations of scale inhibitors in produced water creates uncertainty, thus making it challenging to assess the effectiveness of a polymeric inhibitor squeeze treatment over time.

The present application addresses these and other challenges related to measuring and analyzing polymeric scale inhibitors in water.

### SUMMARY OF THE DISCLOSURE

In a first aspect, a method of analyzing the residual of a polymeric scale inhibitor in produced water is provided. In the method, an amount of SO₄²⁻ is measured in a sample of produced water comprising a polymeric scale inhibitor, where the polymeric scale inhibitor comprises 80-82 mol % of 2-acrylamino-2-methylpropane sulfonic acid (AMPS). A barium salt is introduced into the sample of produced water, and the barium salt removes SO₄²⁻ from the produced water sample resulting in a low SO₄²⁻ produced water sample. The low SO₄²⁻ produced water sample is then analyzed, where in the analysis: a sulfur level in the low SO₄²⁻ produced water sample is determined, and a thermodynamic scale prediction is performed to calculate the amount of SO₄²⁻ ions remaining in the low SO₄²⁻ produced water sample. A concentration of the polymeric scale inhibitor in the produced water sample is then determined based on an inductively couple plasma (ICP) analysis.

In another aspect, the barium salt is barium chloride, barium chloride dehydrate, barium acetate, barium bicarbonate, or barium nitrate or a combination thereof.

In another aspect, the polymeric scale inhibitor further comprises: 2-18 mol % of a secondary monomeric unit selected from the group consisting of N-vinyl formamide, N-vinyl pyrrolidone, and diallyl dimethyl ammonium chloride; and 2-18 mol % of a tertiary monomeric unit selected from the group consisting of acrylic acid, methacrylic acid, esters of acrylic acid or methacrylic acid with an alcohol having 1 to 4 carbon atoms, and carboxyethyl acrylate.

In another aspect, the polymeric scale inhibitor has a weight average molecular weight between approximately 300 kDa and approximately 1200 kDa.

In another aspect, the method further includes determining whether the concentration of the polymeric scale inhibitor in the produced water sample is at or below a minimum inhibitor concentration (MIC).

In another aspect, the barium salt is introduced into the sample of produced water at a temperature in the range of approximately 10-50° C. and at a pressure in the range of approximately 15-30 psi. In a further aspect, wherein the barium salt is introduced into the sample of produced water at a temperature of approximately 25° C. and at a pressure of approximately 15 psi.

In another aspect, the amount of barium salt introduced into the sample of produced water is proportionate to the measured amount of SO₄²⁻ in the sample of produced water.

In another aspect, the step of introducing the barium salt comprises: precipitating out BaSO₄ formed as a result of a reaction between the barium salt and the SO₄²⁻ in the produced water sample, and filtering the BaSO₄ out of the produced water sample to form the low-SO₄²⁻ produced water sample.

In a second aspect, a method of analyzing the residual of a polymeric scale inhibitor in water containing SO₄²⁻ is provided. In the method, an amount of SO₄²⁻ in a sample of the water is measured, and the water further comprises a polymeric scale inhibitor comprising 2-acrylamino-2-methylpropane sulfonic acid (AMPS). A salt is introduced into the sample of water, where the salt is BaCl₂, SrCl₂, or CaCl₂, and where the salt removes SO₄²⁻ from the water sample resulting in a low SO₄²⁻ water sample. The low SO₄²⁻ water sample is then analyzed, where in the analysis: a sulfur level in the low SO₄²⁻ water sample is determined, and a thermodynamic scale prediction is performed to calculate the amount of SO₄²⁻ ions remaining in the low SO₄²⁻ water sample. A concentration of the polymeric scale inhibitor in the water sample is then determined based on an inductively couple plasma (ICP) analysis.

In another aspect, the polymeric scale inhibitor comprises: 80-82 mol % of 2-acrylamino-2-methylpropane sulfonic acid (AMPS); 2-18 mol % of a secondary monomeric unit selected from the group consisting of N-vinyl formamide, N-vinyl pyrrolidone, and diallyl dimethyl ammonium chloride; and 2-18 mol % of a tertiary monomeric unit selected from the group consisting of acrylic acid, methacrylic acid, esters of acrylic acid or methacrylic acid with an alcohol having 1 to 4 carbon atoms, and carboxyethyl acrylate.

In another aspect, the step of introducing the salt comprises: precipitating out a sulfate compound formed as a result of a reaction between the salt and the SO₄²⁻ in the water sample, wherein the sulfate compound is BaSO₄, SrSO₄, or CaSO₄, and filtering the sulfate compound out of the water sample to form the low-SO₄²⁻ water sample.

Any combinations of the various embodiments and implementations disclosed herein can be used. These and other aspects and features can be appreciated from the following description of certain embodiments and the accompanying drawings and claims.

### BRIEF DESCRIPTION OF THE DRAWINGS

FIG. 1 displays a flow diagram of an exemplary method of analyzing the residual of a polymeric scale inhibitor in produced water in accordance with one or more embodiments.

FIG. 2 displays a diagram of an exemplary process for reducing SO₄²⁻ in a produced water sample in accordance with one or more embodiments.

### DETAILED DESCRIPTION OF CERTAIN EMBODIMENTS OF THE DISCLOSURE

In accordance with one or more embodiments, disclosed herein are methods of analyzing the residual of a polymeric scale inhibitor in produced water. As mentioned previously, inhibition of iron sulfide mineral scale formation in oil and gas wells is notoriously difficult. However, new polymeric scale inhibitors have been developed for inhibiting this type of scale formation, including polymeric scale inhibitor that comprises 2-acrylamino-2-methylpropane sulfonic acid (AMPS). These AMPS-based polymeric scale inhibitors show superior adsorption characteristics on rocks and their subsequent release behavior allows for scale inhibition over extended time periods respectively at a significant volume of coreflood fluids. They are especially suited for downhole application via field scale squeeze treatments. However, while AMPS-based polymeric scale inhibitors alone are easily analyzed by Inductively Couple Plasma (ICP) through sulfur detection or similarly methods, when AMPS-based inhibitors are in produced water, the interference of sulfur from SO₄²⁻ already present in the produced water significantly interferes with the residual analysis of this polymeric scale inhibitor using ICP analysis.

The present methods for analyzing the residual of a polymeric scale inhibitor in produced water address the problem of sulfur interference and other challenges related to analyzing polymeric scale inhibitors, and in particular AMPS-based inhibitors.

The present methods are described in further detail below. Further, as used in the present application, the term “approximately” when used in conjunction with a number refers to any number within 5% of the referenced number, including the referenced number. As used herein, “produced water” generally refers to water that is trapped in underground formations and is brought to the surface during oil and gas exploration and production. As such, “produced water” is generally water that is produced as a byproduct during the extraction of oil and natural gas. However, it should be understood that although the present methods are described herein as being performed on produced water samples, in at least one embodiment, the present methods can be performed using other types of water with high sulfate levels.

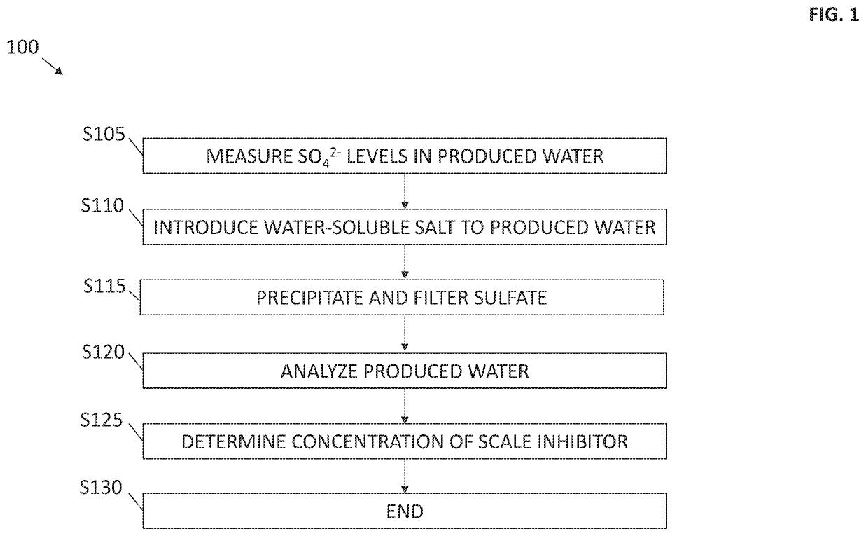

FIG. 1 displays a flow diagram of an exemplary method of analyzing the residual of a polymeric scale inhibitor in produced water in accordance with one or more embodiments. With reference to FIG. 1, the method 100 begins at step S105, where the amount of SO₄²⁻ in a sample of produced water comprising a polymeric scale inhibitor is measured. In one or more embodiments, the amount or concentration of SO₄²⁻ in the produced water is measured analyzing the concentration of sulfur in the produced water via ion chromatography, for example, and then calculating the SO₄²⁻ level based on the concentration of sulfur.

As mentioned above, the polymeric scale inhibitor in the produced water sample comprises 2-acrylamino-2-methylpropane sulfonic acid (AMPS). Specific embodiments of these AMPS-based polymeric scale inhibitors are provided in U.S. patent application Ser. No. 16/804,876, which is hereby incorporated by reference in its entirety. For example, in one or more embodiments, the polymeric scale inhibitor comprises 80-82 mol % of AMPS. In at least one embodiment, in addition to the 80-82 mol % of AMPS, the polymeric scale inhibitor further comprises: 2-18 mol % of a second monomeric unit selected from N-vinyl formamide, N-vinyl pyrrolidone, and diallyl dimethyl ammonium chloride; and 2-18 mol % of a third monomeric unit selected from acrylic acid, methacrylic acid, esters of acrylic acid or methacrylic acid with an alcohol having 1 to 4 carbon atoms, and carboxyethyl acrylate. In one or more embodiments, the polymeric scale inhibitor has a weight average molecular weight between approximately 300 kDa and 1200 kDa.

With continued reference to FIG. 1, at step S110, a water-soluble salt is introduced into the sample of produced water, where the water-soluble salt can later remove SO₄²⁻ from the produced water sample (e.g., via chemical reaction) resulting in a low-SO₄²⁻ produced water sample (see step S115). In one or more embodiments, the water-soluble salt is a water-soluble barium salt, such as barium chloride (BaCl₂), barium chloride dehydrate (BaCl₂—2H₂O), barium acetate (Ba(CH₃COO)₂), barium bicarbonate (Ba(HCO₃)₂), or barium nitrate (Ba(NO₃)₂), for example. In at least one embodiment, other water-soluble salts can be used instead of barium salts, such as strontium chloride (SrCl₂) or calcium chloride (CaCl₂).

At step S115, the SO₄²⁻ (specifically, the resulting sulfate compounds) in the produced water is precipitated out and then filtered out of the sample of produced water to form a low-SO₄²⁻ produced water sample. More specifically, the introduction of barium salts (or SrCl₂ or CaCl₂) results in a chemical reaction in the produced water sample in which the salt reacts with the sulfate to form barium sulfate (BaSO₄) precipitates, or in the case of introduction SrCl₂ or CaCl₂ salts, strontium sulfate (SrSO₄) or calcium sulfate (CaSO₄) precipitates, respectively. These sulfate precipitates can then be removed from the water sample via filtration to form a low-SO₄²⁻ produced water sample.

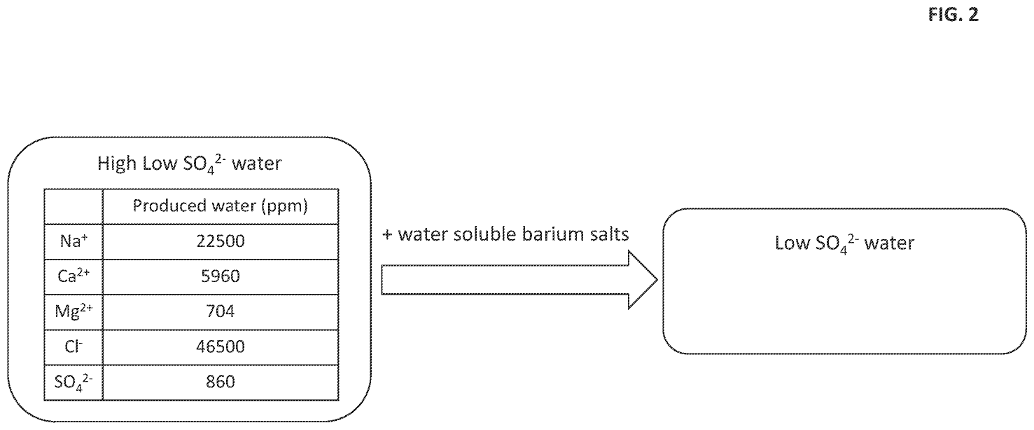

An exemplary diagram of adding a water-soluble salt (e.g., water-soluble barium salt) to reduce SO₄²⁻ levels in a produced water sample, as done in steps S110 and S115, is shown in FIG. 2. As shown in FIG. 2, the produced water can be comprised of several different ions including sodium (Na⁺), calcium (Ca²⁺), magnesium (Mg²⁺), chlorine (Cl⁺), and sulfate (SO₄²⁻) ions. In one or more embodiments, produced water comprising the AMPS-based polymeric scale inhibitor, as in the present method, has high levels of sulfate (SO₄²⁻), which stems from sulfur present in the produced water from other sources.

As such, conventional methods (e.g., ICP-MS, ICP-OES) alone cannot determine the amount of the AMPS-based polymeric scale inhibitor from the produced water sample because the sulfate levels in the produced water interfere with sulfur measurements that are typically used to determine the amounts of the inhibitor in the produced water. In contrast, in the present method, the introduction of the water-soluble salts (e.g., water-soluble barium salts), helps to remove that interference.

For example, the addition of one or more barium salts to the produced water sample forms BaSO₄ scales (BaSO₄ precipitation) in the produced water as a result of a reaction between barium ions and sulfate ions. Similarly, in other embodiments, strontium ions or calcium ions from strontium chloride (SrCl₂) or calcium chloride (CaCl₂) salts, respectively, similarly react with sulfate in the produced water sample to form SrSO₄ or CaSO₄ precipitates. In one or more embodiments, these sulfate precipitates can be removed from the produced water sample via filtration. Accordingly, the addition of barium salts (or SrCl₂ or CaCl₂) to the produced water sample results in the removal of sulfates from the produced water resulting in a low-SO₄²⁻ produced water sample (FIG. 2), and thereby allowing for an effective analysis of the amount of sulfur in the residual of the polymeric scale inhibitor in the produced water. The solubility of BaSO₄ in water is less than that of SrSO₄ and CaSO₄. Specifically, the solubility order of these sulfate precipitates is as follows: CaSO₄>SrSO₄>BaSO₄. As such, in one or more embodiments, introduction of barium salts can remove sulfate to lower levels (i.e., remove more sulfate) than SrCl₂ or CaCl₂.

In one or more embodiments, the water-soluble salt (e.g., barium salt, SrCl₂, CaCl₂) is introduced into the sample of produced water (at step S110) at a temperature in the range of approximately 10-50° C. and at a pressure in the range of approximately 15-30 psi. In at least one embodiment, the water-soluble salt (e.g., barium salt, SrCl₂, CaCl₂) is introduced into the produced water sample (at step S110) at a temperature of approximately 77° F. (25° C.) and at a pressure of approximately 15 psi. In at least one embodiment, when the water-soluble salt used is CaCl₂, CaCl₂ can be introduced into the produced water at a higher temperature range, such as in the range of approximately 10-95° C.

In one or more embodiments, the amount of water-soluble salt introduced into the produced water sample is proportionate to the measured amount of SO₄²⁻ in the sample of produced water from step S105. In other words, the amount of water soluble salt added to the produced water at step S110 depends on the amount of SO₄²⁻ present in the produced water. For example, in the case of a barium salt being added at step S110, the equilibrium amount of barium salt can be calculated with the reaction Ba²⁺+SO₄²⁻→BaSO₄. In at least one embodiment, approximately 1.5-3 times the equilibrium amount of barium salt is added into the produced water to result in full or substantially full precipitation of SO₄²⁻. For instance, in such an embodiment, when approximately 1.5-3 times of the equilibrium amount of barium salt is added to the produced water over a period of approximately 12-24 hours, the SO₄²⁻ can be reduced to a neglectable level.

In one or more embodiments, whether the amount of water soluble salt added to the produced water results in full or substantially full precipitation of sulfate can be predicted by model as exemplified in Table 3 below. Moreover, whether full or substantially full precipitation has occurred can be analyzed or verified by ICP methods, for example.

With continued reference to FIG. 1, at step S120, the low SO₄²⁻ produced water sample is analyzed. In one or more embodiments, the low SO₄²⁻ produced water sample can be analyzed to determine the amount of sulfate (SO₄²⁻) remaining in the produced water sample after precipitation (e.g., BaSO₄, CaSO₄, or SrSO₄ precipitation) and the remaining amount of sulfur in the produced water sample after BaSO₄, CaSO₄, or SrSO₄ precipitation. Because the sulfate precipitation removes the influence of sulfate on the analysis, the amount of sulfur detected in the produced water after precipitation correlates to the sulfur of the AMPS-based polymeric scale inhibitor.

In at least one embodiment, as part of step S120, thermodynamic scale prediction is undertaken to calculate the amount of SO₄²⁻ ions remain in the produced water after adding the water-soluble salt and the subsequent precipitation of BaSO₄, CaSO₄, or SrSO₄. In an exemplary embodiment, the thermodynamic scale prediction can be performed in part using one or more software programs, such as the ScaleSoftPitzer program. The scale prediction calculations provide values for the supersaturation ratio (SR), which is a parameter indicating the thermodynamic driving force for the formation of scales (e.g., BaSO₄, CaSO₄, or SrSO₄ scales) and also the mass of scale precipitate. When the SR=1, the scaling formation and dissolution is equivalent, and no more scales will form thermodynamically. The concentration of the scaling cation and anion can be calculated when SR reaches the value of 1. For example, in embodiments in which the salt introduced into the at step S110 is a barium salt, when the SR is 1, the concentration of the scaling cation (Ba²⁺) and anion (SO₄²⁻) can be calculated.

At step S125, the concentration of the AMPS-based polymeric scale inhibitor is determined. In one or more embodiments, the concentration of the AMPS-based polymeric scale inhibitor is determined based on the amount of sulfur detected in the produced water after sulfate precipitation, which correlates to the sulfur of the AMPS-based polymeric scale inhibitor. In one or more embodiments, the concentration of the AMPS-based polymeric scale inhibitor is determined via ICP methods through sulfur detection, such as ICP-OES and ICP-MS. In at least one embodiment, as part of the step of determining the concentration of the AMPS-based polymeric scale inhibitor, a determination is made as to whether the concentration of the polymeric scale inhibitor is at or below a minimum inhibitor concentration (MIC).

At step S130 the method ends.

The present methods provide enhanced iron sulfide scale inhibitor residual analyses as compared with current practices. Specifically, the present method provides a more effective and accurate way to analyze the residual of an AMPS-based polymeric scale inhibitor in a produced fluid by effectively removing the influence of SO₄²⁻ on the residual analysis using water-soluble salts. This allows the quantitative determination the AMPS-based scale inhibitor in the produced water via real-time ICP through sulfur detection to be more feasible, as the ICP equipment can include a mass spectrometer detector (ICP-MS) or optical emission spectroscopic detector (ICP-OES). An accurate quantitative measurement of the AMPS-based scale inhibitor, including determining whether the concentration of the inhibitor is at or below a MIC, is vital for achieving the required longevity for scale inhibitor treatments and preventing wells and downhole tubing from scaling.

The present method also provides enhanced sensitivity for measuring scale inhibitor residue, as it allows for a lower limit of detection and quantification. More specifically, ICP or HPLC methods have been previously used to measure the residual scale inhibitors in the produced water. However, both methods alone cannot accurately measure the AMPS-based scale inhibitor described herein accurately. As such, methods of the present application provide a lower limit of detection of the AMPS-based inhibitor and more accurate quantification of the AMPS-based inhibitor. Finally, the present methods for analyzing the residual of the polymeric scale inhibitor is faster than existing analysis methods and eliminates the need for using more arduous techniques, such as solid phase extraction.

These and other advantages of the present methods are further understood and described in the following example.

### Example

In the present example, the polymeric scale inhibitor is composed of 80-82 mol % 2-acrylamido-2-methylpropane sulfonic acid (AMPS). The sulfur content in this polymeric scale inhibitor is about 6.2%. The concentration of sulfur contributed by this polymeric scale inhibitor is shown in Table 1 at 3 different corresponding concentrations of the polymeric scale inhibitor in produced water.

TABLE 1
Concentration of sulfur from the polymeric scale inhibitor.

| Concentration of | |
|---|---|
| polymeric scale | Corresponding concentration of sulfur |
| inhibitor in produced | contributed by polymeric scale |
| water (ppm) | inhibitor in produced water (ppm) |
| 20 | 1.24 |
| 50 | 3.1 |
| 100 | 6.2 |

The synthetic brine chemistry of a produced water is shown in Table 2.

TABLE 2
Water chemistry of a produced water.

| Ion | Produced water (ppm) |
|---|---|
| Na⁺ | 22500 |
| Ca²⁺ | 5960 |
| Mg²⁺ | 704 |
| CI⁻ | 46500 |
| SO₄²⁻ | 860 |

The concentration of SO₄²⁻ in the produced water is 860 ppm (Table 2), and the sulfur contributed by the produced water is 287 ppm. Conventionally, it would be difficult to analyze the concentration of 20 ppm polymeric scale inhibitor (1.24 ppm S) in the produced water having 287 ppm sulfur using an ICP sulfur analysis due to the interference of the large amount of SO₄²⁻ in the produced water.

To overcome this deficiency, the sulfates in the produced water are removed by adding one or more water-soluble barium salts to the produced water sample. BaSO₄ scales are formed in the produced water primarily due to mixing various types of water-soluble barium salts, such as barium chloride (BaCl₂), barium chloride dehydrate (BaCl₂—2H₂O), barium acetate (Ba(CH₃OOO)₂), barium bicarbonate (Ba(HCO₃)₂), or Ba nitrate (Ba(NO₃)₂) with the produced water that contains high concentration of sulfate ions.

Scale prediction is then undertaken to calculate the amount of SO₄²⁻ ion remaining in the produced water after adding the barium salt (e.g., BaCl₂). Specifically, a thermodynamic scale prediction is performed using the ScaleSoftPitzer program, and the scale prediction calculations provided values for the Supersaturation Ratio (SR). When the SR=1, the scaling formation and dissolution is equivalent, and thus scale will no longer form thermodynamically. The concentration of the scaling cation (Ba²⁺) and anion (SO₄²⁻) is then calculated when SR reaches the value of 1. The calculated amount of sulfate remaining in the produced water and BaSO₄ precipitation after adding different amount of BaCl₂—2H₂O at 77° F. and 15 psi are shown in Table 3.

As shown in Table 3, the remaining sulfur contributed by SO₄²⁻ in produced water was reduced to 0.01 ppm by adding 1.60 mg of BaCl₂—2H₂O in 100 mL of produced water (see last row), where the interference of sulfur contained within the polymeric scale inhibitor analysis form the sulfur present in SO₄²⁻ in the produced water due to high SO₄²⁻ in produced water can be ignored. The sulfur analysis by ICP is then applied to analyze the residual polymeric scale inhibitor-namely, to determine a concentration of the polymeric scale inhibitor in the produced water sample.

TABLE 3
calculated sulfate remaining in produced water and BaSO₄ precipitation
after adding BaCl₂•2H₂O at 77° F. and 15 psi.

| | | | | Remaining | Remaining |
|---|---|---|---|---|---|
| | | | | SO₄ in | sulfur in |
| | BaCl₂•2H₂O | | Removed | produced | produced |
| SO₄²⁻ in | added in | Ba | SO₄ by | water after | water after |
| produced | 100 ml | concentration | BaSO₄ | fully BaSO₄ | fully BaSO₄ |
| water | produced | in water | precipitation | precipitation | precipitation |
| (ppm) | water (mg) | (ppm) | (ppm) | (ppm) | (ppm) |
| 860 | 0.2 | 1125 | 1909.1 | ~73 | 23.84 |
| 860 | 0.4 | 2250 | 2086.8 | 0.183 | 0.06 |
| 860 | 0.8 | 4500 | 2087.0 | 0.064 | 0.02 |
| 860 | 1.6 | 9000 | 2087.2 | 0.028 | 0.01 |

Although much of the foregoing description has been directed to methods for analyzing the residual of a polymeric scale inhibitor in water, the methods disclosed herein can be similarly deployed and/or implemented in scenarios, situations, and settings far beyond the referenced scenarios. It should be further understood that any such implementation and/or deployment is within the scope of the systems and methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms ““including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all of the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A method of analyzing the residual of a polymeric scale inhibitor in produced water, comprising:
measuring an amount of SO₄²⁻ in a sample of produced water comprising a polymeric scale inhibitor, wherein the polymeric scale inhibitor comprises 80-82 mol % of 2-acrylamino-2-methylpropane sulfonic acid (AMPS);
introducing a barium salt into the sample of produced water, and wherein the barium salt removes SO₄²⁻ from the produced water sample resulting in a low SO₄²⁻ produced water sample;
analyzing the low SO₄²⁻ produced water sample, wherein the analysis includes:
determining a sulfur level in the low SO₄²⁻ produced water sample, and performing a thermodynamic scale prediction to calculate the amount of SO₄²⁻ ions remaining in the low SO₄²⁻ produced water sample; and
determining a concentration of the polymeric scale inhibitor in the produced water sample based on an inductively couple plasma (ICP) analysis.

2. The method of claim 1, wherein the barium salt is barium chloride, barium chloride dehydrate, barium acetate, barium bicarbonate, or barium nitrate or a combination thereof.

3. The method of claim 1, wherein the polymeric scale inhibitor further comprises:
2-18 mol % of a secondary monomeric unit selected from the group consisting of N-vinyl formamide, N-vinyl pyrrolidone, and diallyl dimethyl ammonium chloride; and
2-18 mol % of a tertiary monomeric unit selected from the group consisting of acrylic acid, methacrylic acid, esters of acrylic acid or methacrylic acid with an alcohol having 1 to 4 carbon atoms, and carboxyethyl acrylate.

4. The method of claim 1, wherein the polymeric scale inhibitor has a weight average molecular weight between approximately 300 kDa and approximately 1200 kDa.

5. The method of claim 1, further comprising:
determining whether the concentration of the polymeric scale inhibitor in the produced water sample is at or below a minimum inhibitor concentration (MIC).

6. The method of claim 1, wherein the barium salt is introduced into the sample of produced water at a temperature in the range of approximately 10-50° C. and at a pressure in the range of approximately 15-30 psi.

7. The method of claim 6, wherein the barium salt is introduced into the sample of produced water at a temperature of approximately 25° C. and at a pressure of approximately 15 psi.

8. The method of claim 1, wherein the amount of barium salt introduced into the sample of produced water is proportionate to the measured amount of SO₄²⁻ in the sample of produced water.

9. The method of claim 1, wherein the step of introducing the barium salt comprises:
precipitating out BaSO₄ formed as a result of a reaction between the barium salt and the SO₄²⁻ in the produced water sample, and
filtering the BaSO₄ out of the produced water sample to form the low-SO₄²⁻ produced water sample.

10. A method of analyzing the residual of a polymeric scale inhibitor in water containing SO₄²⁻, comprising:
measuring an amount of SO₄²⁻ in a sample of the water, wherein the water further comprises a polymeric scale inhibitor, and wherein the polymeric scale inhibitor comprises 2-acrylamino-2-methylpropane sulfonic acid (AMPS);
introducing a salt into the sample of water, wherein the salt is BaCl₂, SrCl₂, or CaCl₂, and wherein the salt removes SO₄²⁻ from the water sample resulting in a low SO₄²⁻ water sample;
analyzing the low SO₄²⁻ water sample, wherein the analysis includes:
determining a sulfur level in the low SO₄²⁻ water sample, and performing a thermodynamic scale prediction to calculate the amount of SO₄²⁻ ions remaining in the low SO₄²⁻ water sample; and
determining a concentration of the polymeric scale inhibitor in the water sample based on an inductively couple plasma (ICP) analysis.

11. The method of claim 10, wherein the polymeric scale inhibitor comprises:
80-82 mol % of 2-acrylamino-2-methylpropane sulfonic acid (AMPS);
2-18 mol % of a secondary monomeric unit selected from the group consisting of N-vinyl formamide, N-vinyl pyrrolidone, and diallyl dimethyl ammonium chloride; and
2-18 mol % of a tertiary monomeric unit selected from the group consisting of acrylic acid, methacrylic acid, esters of acrylic acid or methacrylic acid with an alcohol having 1 to 4 carbon atoms, and carboxyethyl acrylate.

12. The method of claim 10, wherein the polymeric scale inhibitor has a weight average molecular weight between approximately 300 kDa and approximately 1200 kDa.

13. The method of claim 10, further comprising:
determining whether the concentration of the polymeric scale inhibitor in the water sample is at or below a minimum inhibitor concentration (MIC).

14. The method of claim 10, wherein the amount of salt introduced into the sample of water is proportionate to the measured amount of SO₄²⁻ in the sample of water.

15. The method of claim 10, wherein the salt is introduced into the sample of water at a temperature in the range of approximately 10-50° C. and at a pressure in the range of approximately 15-30 psi.

16. The method of claim 15, wherein the salt is introduced into the sample of water at a temperature of approximately 25° C. and at a pressure of approximately 15 psi.

17. The method of claim 10, wherein the step of introducing the salt comprises:
precipitating out a sulfate compound formed as a result of a reaction between the salt and the SO₄²⁻ in the water sample, wherein the sulfate compound is BaSO₄, SrSO₄, or CaSO₄, and
filtering the sulfate compound out of the water sample to form the low-SO₄²⁻ water sample.
